(19) United States
(12) Patent Application Publication
(10) Pub. No.: US 2011/0264219 A1
(43) Pub. Date: Oct. 27, 2011

(54) SPACER FOR SPINAL FUSION

(76) Inventor: David P. Rouben, Louisville, KY (US)

(21) Appl. No.: 12/765,282

(22) Filed: Apr. 22, 2010

(51) Int. Cl. A61F 2/44

(52) U.S. Cl. 623/17.16

(57) ABSTRACT

A spacer for use in a spinal fusion procedure includes a hollow, elongated body including a central portion adapted to contact the medullary bone portion of the vertebrae and nubs projecting in the elongated direction from the central portion and adapted to contact the apophyseal rings of the vertebrae.

### BACKGROUND

The present invention relates to a spacer used in a spinal fusion procedure to replace a damaged intervertebral disc between vertebrae.

Sometimes, due to age, illness, genetics, or injury, the disc between two vertebrae in the spine becomes herniated or otherwise structurally damaged. When the disc is damaged, it may no longer maintain a desired spacing or cushion between the two adjacent vertebrae, resulting in pinched nerves and pain. The damaged disc then may need to be removed. A standard procedure involves removing the damaged disc, separating (or distracting) the affected vertebrae to provide a space between them, and then fusing the two adjacent spaced-apart vertebrae together, with bone growing through and around the spacer and rigidly tying the vertebrae above and below the spacer together. The surgical procedure may be performed anteriorly (from the front of the patient), posteriorly (from the back of the patient), or laterally (from the side of the patient), and the spacer often is delivered and inserted between the affected vertebrae with the aid of a cannula (which is a tube) and/or a delivery wand.

Bones in general (including vertebral bones) have a dense, outer surface called the cortical bone (or cortical portion), and a soft, spongy, inner portion called the medullary bone (or cancellous bone). The cortical bone forms a dense shell around the more delicate medullary bone. In a plan view of a vertebra, the cortical bone forms an outer dense, bony ring, referred to as the apophyseal ring, which surrounds the softer medullary bone. Considering the vertebra in the upright position, with the spinal cord extending vertically, the medullary bone portion typically has a horizontal diameter ranging from 25 to 40 mm, and the apophyseal ring has a horizontal width or thickness in the range of 1.5 mm to 3 mm, which adds 3 mm to 6 mm to the diameter of the vertebra, making the total diameter of a typical vertebra in the range of 28 to 46 mm.

Looking at a vertical (sagittal cut) section through the vertebra, there is a contour, with the height of the vertebra at the peripheral apophyseal ring being substantially greater than the height at the center of the medullary portion. The medullary bone has a concave shape, which creates a convex, elliptical space between adjacent vertebrae in the area of the medullary bone portion.

Most prior disc spacers, once installed, lie entirely within that elliptical space. This often creates a problem in that the soft, medullary bone may not provide adequate support to the spacer, allowing the spacer to sink into this softer medullary bone structure (implant subsidence), so that the spacer is not able to perform its intended function of maintaining a desired spacing between the vertebrae, which again may result in pinched nerves and pain (the very problems the fusion procedure was intended to solve).

Some known spacers, such as the Novel® TL Spacer System offered by Alphatec Spine Inc of Carlsbad, Calif., are elongated and have wedge-shaped ends, gradually tapering from a lesser height at the very end to a greater height progressing inwardly (also referred to as “bullet nose” in the company literature) to facilitate insertion of the spacer (cage) between the vertebrae. Of course, this same wedge shape at the end of the spacer also can facilitate the expulsion of the spacer out the other side of the gap between the affected vertebrae or cause the end of the spacer to migrate into the central interior of the vertebra, into the area of the soft, medullary bone, and away from the apophyseal ring.

Other known spacers, such as the Clydesdale® spinal system made by Medtronic, are long enough to span the entire horizontal diameter of the space between the vertebrae between which the spacer is to be installed. However, these spacers are too large to be inserted from a posterior approach, anterior approach, or a posterior-lateral approach, and thus are installed laterally. The lateral approach precludes the installation of the spacer (cage) between the Lumbar 5 and Sacral 1 vertebrae (L5-S1) due to interference with the iliac crest (pelvis bone), and this is the area in which a large portion of spinal fusions occur.

### SUMMARY

One embodiment of the present invention provides a spacer in the shape of an elongated body. The central portion of the elongated body has a length which spans the medullary bone portion of the vertebra. Nubs project from each end of the central portion of the elongated body. The nubs define support surfaces which rest on the cortical or apophyseal ring, using the structurally strong perimeter of the vertebrae to support the central, softer cancellous bone portion. The connection point between the nub and the central portion of the elongated body preferably defines a shoulder designed to abut the apophyseal ring at its interface with the medullary bone of the vertebra so as to provide a stop which helps ensure proper placement of the spacer during the installation process and helps prevent the spacer from shifting relative to the vertebrae after it is installed.

### BRIEF DESCRIPTION OF THE DRAWINGS

FIG. 1 is a perspective view of a spacer made in accordance with the present invention;

FIG. 2 is a right end view of the spacer of FIG. 1;

FIG. 3 is a plan view of the spacer of FIG. 1;

FIG. 4 is a front view of the spacer of FIG. 1;

FIG. 5 is a front (anterior), perspective view, partially in section, of two vertebrae stacked one above the other as found in a human spinal column;

FIG. 6 is a plan view of a human vertebra with the spacer of FIG. 1 being inserted transforaminally (posterior-lateral approach) between two vertebrae;

FIG. 7 is the same view as FIG. 6 but with the spacer essentially fully inserted though not yet in its final position;

FIG. 8 is the same view as FIG. 7 but with the spacer in its final position;

FIG. 9 is a front, perspective view, partially in section, of the vertebrae of FIG. 5, with the spacer of FIG. 8 in its final installed position between the vertebrae;

FIG. 10 is a side perspective view of the vertebrae and spacer of FIG. 9;

FIG. 11 is a left end view of the spacer of FIG. 1;

FIG. 12 is an end view, similar to FIG. 11, but for another embodiment of a spacer made in accordance with the present invention;

FIG. 13 is a back (posterior) view of the vertebrae showing a facectomy to provide posterior-lateral access to the inter-disc space.

FIG. 14 is a broken away, enlarged view of the right end of the spacer of FIG. 4;

FIG. 15 is view similar to FIG. 14, but for another embodiment of a spacer; and

FIG. 16 is a broken way, perspective view of the right rear portion of another embodiment of a spacer.

### DESCRIPTION

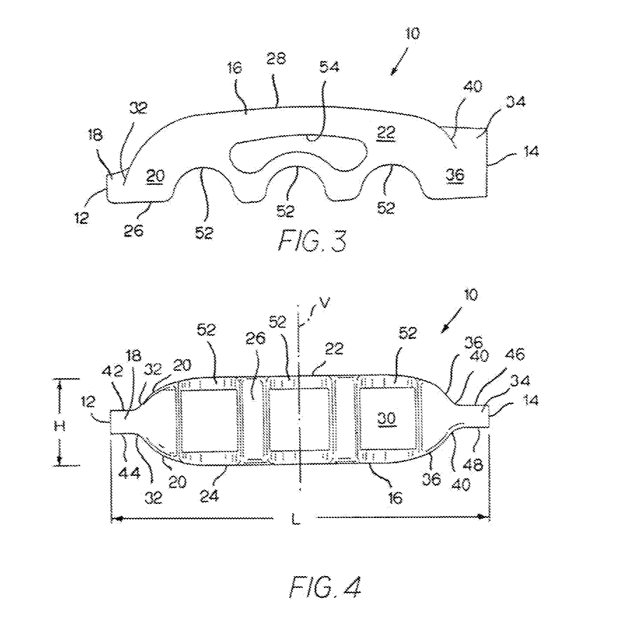

FIGS. 1-11 and 13 show an embodiment of a spacer 10, which is used to replace a damaged disc between two adjacent vertebrae, as described in more detail later.

Referring to FIGS. 1-4, the spacer 10 is an elongated, substantially hollow body having a first end 12, a second end 14, and a central portion 16 between the first and second ends 12, 14 respectively. The spacer 10 has a top surface 22, a bottom surface 24, a front surface 26, a rear surface 28, and a hollow interior surface 30. The first end 12 defines a first nub 18 projecting in the elongated direction from the central portion 16, and a first shoulder 20 on both the top and bottom surfaces 22, 24, recessed from the first end 12. A concave inflection point 32 in each of the top and bottom body surfaces 22, 24 defines an intersection between the first nub 18 and the first shoulder 20 of the spacer 10. Similarly, the second end 14 defines a second nub 34 projecting in the elongated direction from the central portion 16 and a second shoulder 36 on both the top and bottom surfaces 22, 24, recessed from the second end 14. A concave inflection point 40 in each of the top and bottom body surfaces 22, 24 defines an intersection between the second nub 34 and the second shoulder 36 of the spacer 10.

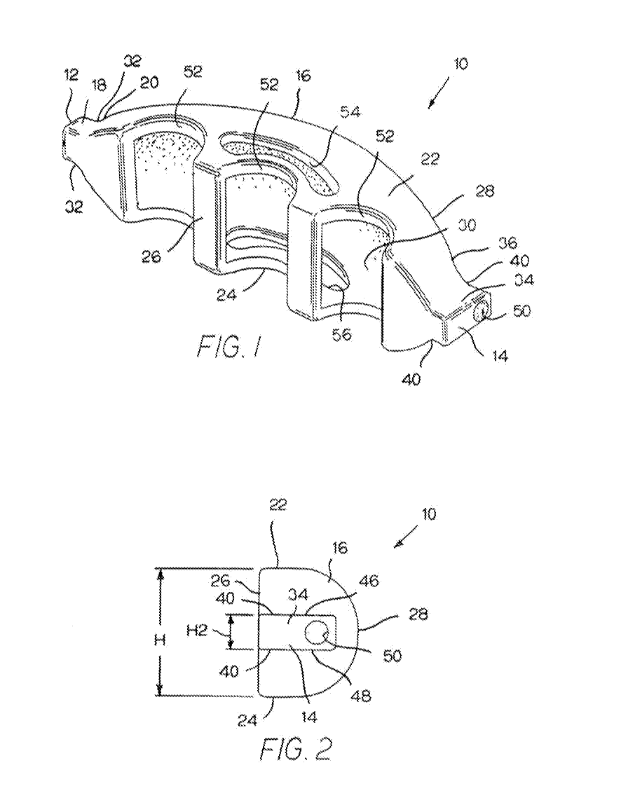

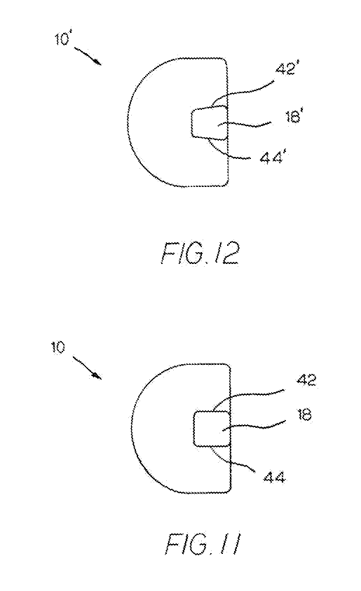

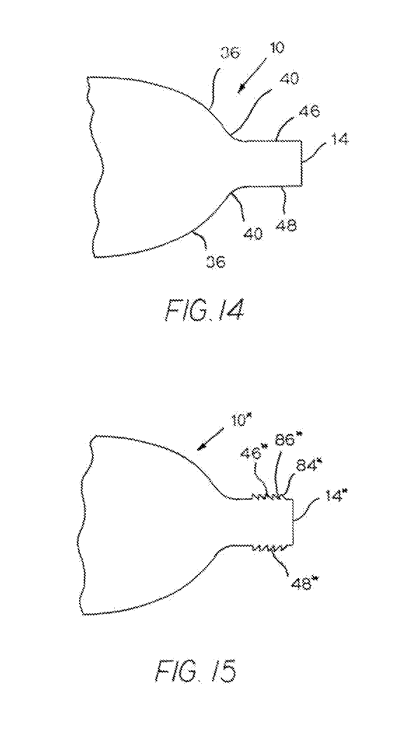

The first nub 18 has substantially flat top and bottom surfaces 42, 44 which lie perpendicular to the vertical axis “V” of the spinal column when the spacer 10 is installed. These surfaces 42, 44 can be seen best in FIG. 11. The second nub 34 also has substantially flat upper and lower surfaces 46, 48, respectively, which lie perpendicular to the vertical axis of the spinal column when the spacer 10 is installed. These surfaces are shown best in FIG. 2. These flat top and bottom surfaces 42, 44, 46, 48 may be modified as shown in FIG. 15, where the surfaces 46*, 48* have a roughened surface, with short ridges 84* and recesses 86* to promote gripping, if desired.

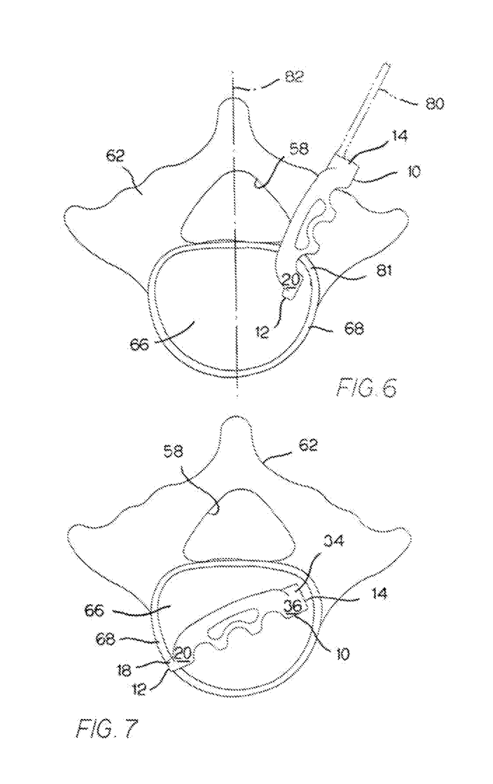

In the embodiment of FIG. 1, the second nub 34 is wider in the front-to-back direction than is the first nub 18 in order to accommodate a means for releasably securing the spacer 10 to an installation wand 80 (See FIG. 6) for installation of the spacer 10 into the inter-disc space, as explained later. In this embodiment, the means for releasably securing the spacer 10 to an installation wand 80 is a threaded opening 50 which receives the threaded end of the installation wand 80. Other means may be used to install the spacer 10 into the inter-disc space, such as the use of pincers to lock onto small openings (not shown) also located in the second nub 34, in which case the added width of the second nub 34 may be eliminated.

In this embodiment, the maximum body height “H”, defined as the maximum dimension between the top and bottom surfaces 22, 24 of the spacer 10 (as shown in FIGS. 2 and 4), is less than the total length dimension “L” of the spacer in the horizontal, elongated direction, defined as the dimension from the first end 12 to the second end 14 (also shown in FIG. 4). The length of each nub 18, 34 in the horizontal elongated direction is from 2 mm to 5 mm. In this embodiment, the maximum nub height H2 (shown in FIG. 2), defined as the maximum dimension between the top and bottom surfaces of the nub, does not exceed 8 mm and preferably is within the range of 2-8 mm. Also, in this embodiment, the maximum body height “H” is at least four millimeters greater than the maximum heights of the nubs 18, 34.

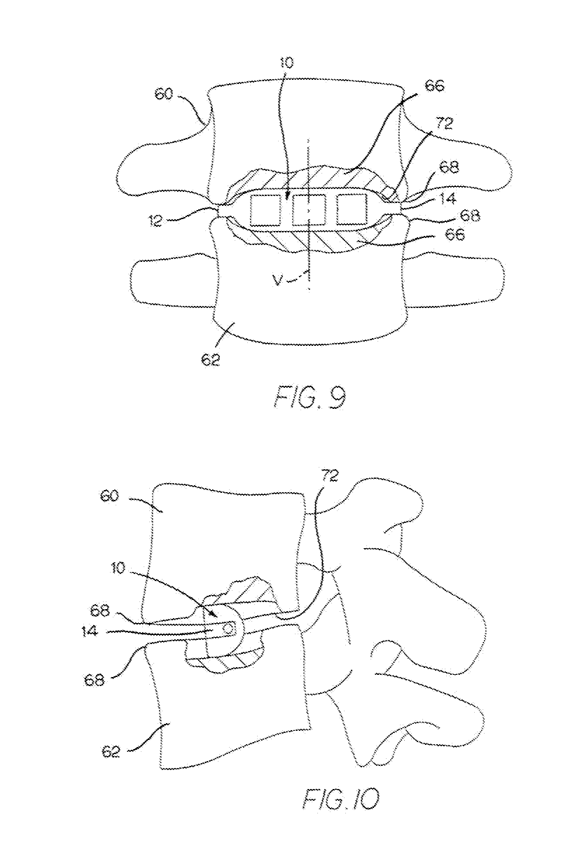

It should be noted that, in this embodiment, the maximum nub heights of the first and second nubs 18, 34 are substantially equal, the first and second nub top surfaces 42, 46 are substantially coplanar and the first and second nub bottom surfaces 44, 48 are also substantially coplanar, with the plane of the top surfaces 42, 46 being substantially parallel to the plane of the bottom surfaces 44, 48 and both of those planes being perpendicular to an imaginary vertical line “V” (See FIG. 9) extending through the top and bottom surfaces of the spacer 10.

Referring to FIGS. 1, 3, and 4, the front surface 26 of the spacer 10 defines a plurality of U-shaped arches 52 and defines openings through the front surface into the hollow interior 30. There are additional openings 54 (through the top surface 22) and 56 (through the bottom surface 24) into the hollow interior 30 (See FIG. 1). This embodiment 10 has no openings along the rear surface 28 of the spacer 10. This construction creates a cage which is substantially open from the front and from the top and bottom, to be able to receive bone graft material and to encourage bone growth through the spacer 10 to enhance the natural incorporation of the fusion in and around the spacer and the affected vertebrae. At the same time, the closed rear surface 28 prevents the bone graft material from coming out of the back of the spacer 10 as it is being inserted.

While there could be some small open area in the rear surface 28, if desired, it should be minimal (less than 10% of the total rear surface area, or less than 50% of the front open area), and it is preferred that there be no opening through the rear surface 28. As may be appreciated from FIG. 8 which shows the spacer 10 after installation, the absence of an opening in the rear surface 28 prevents the bone graft material from accidentally migrating out through the rear surface 28 of the spacer 10 and into the vertebral canal 58 which houses the spinal cord and the nerve roots inside the vertebral column.

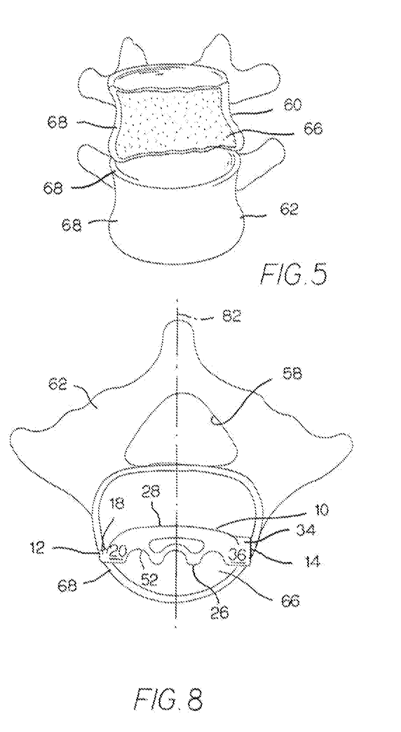

FIG. 5 is a front perspective view, partly in section, of two adjacent, upper and lower vertebrae 60, 62, respectively, showing the dense cortical bone forming the apophyseal ring 68, and the soft, spongy, medullary bone portion 66 (or cancellous bone) encircled by the apophyseal ring 68. The cortical bone forms a compact shell around the more delicate medullary bone. A plan view of a vertebra (See FIG. 8) shows the apophyseal ring 68, with a wall thickness generally in the 1.5 to 3 mm range, surrounding the medullary bone 66, which forms a spongy inner core typically ranging between 25 and 40 mm in diameter.

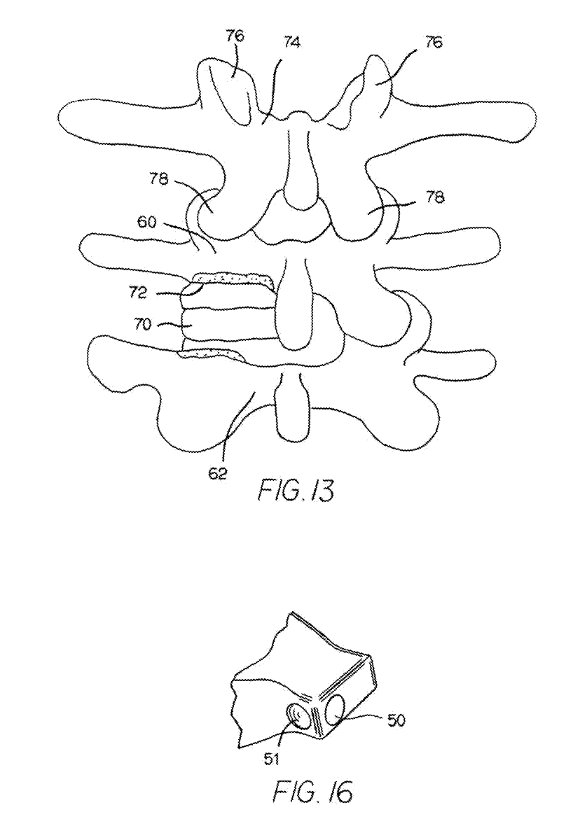

Referring to FIG. 13, a disc 70, made from a cartilaginous material and located in the inter-disc space 72, separates upper and lower adjacent vertebrae 60, 62. As best appreciated in FIG. 9, this inter-disc space 72 is convex in the area of the medullary bone 66, having its greatest height at the center, with the height of the inter-disc space 72 gradually diminishing from the center outwardly toward the apophyseal ring 68, where the height of the space 72 is least. Typically, the inter-disc space at the apophyseal ring 68 is in the range of 2-8 mm, and the maximum inter-disc space at the center of the medullary portion 66 is in the range of 6-16 mm. The apophyseal ring 68 has substantially flat top and bottom surfaces.

FIG. 8 shows an imaginary front-to-back axial plane 82 which bisects the vertebra into first and second substantially mirror-image halves. The preferred approach for the installation of the spacer 10 is by entering into the inter-disc space at a point within 30-45 degrees of the front-to-back axis, which could be an anterior point or a posterior point. If it is a posterior point, the entry into the disc space occurs through the space formed between the caudal aspect of the pedicle of the vertebral body above the disc and the superior aspect of the pedicle of the vertebral body below the disc (the inter-pedicular space).

An abridged summary of an example of a procedure using that approach follows below:

As shown in FIG. 6, in this particular case, the point of entry 81 is on the right side of the imaginary front-to-back vertical axial plane 82.

The first step is to insert pedicle screws (not shown) on the left rear side (the opposite-from-point-of-entry side) of the upper and lower vertebrae 60, 62, and insert a rod along those pedicle screws, but not yet to tighten the set screws fixing the position of the rod relative to the pedicle screws.

Next, insert a guide wire (not shown) to the facet joint posterior to the disc to be fused.

With the aid of dilators, locate the facets and do a complete facetectomy on the point-of-entry side, as shown in FIG. 13, wherein the lower facet of the upper vertebra 60 and the upper facet of the lower vertebra 62 on the point-of-entry side have been resected. For a further reference, the uppermost vertebra 74 has both of its upper facets 76 and the lower facets 78 intact.

Next, cut through the ligamentum flavum, (not shown) which unites the adjacent vertebrae, at the point of entry.

Then, incise the disc space (discectomy) to remove the cartilaginous material (the disc) from the inter-disc space and end plate.

Next, insert the disc Distractor into the disc space to separate the vertebrae to their maximum distraction on the opposite-from-point-of-entry side. On this side, the ligamentum flavum has not been cut through, so it limits the distance the vertebrae can be separated. (On the point-of-entry side, the ligamentum flavum has been cut, which permits the vertebrae to be separated a greater distance on that side.)

Temporarily tighten the set screws to the rod on the opposite-from-point-of-entry side to hold the maximum distraction or separation.

Next, release the Distractor, re-position it closer to the point-of-entry side between the apophyseal rings, and then open the Distractor maximally.

Again temporarily tighten the opposite rod screw connection to maintain the distracted position, and then remove the Distractor.

The Outer Cortical Apophyseal rim may be removed at the point-of-entry to help access the disc space.

Insert bone graft material into the hollow interior of the spacer 10 and then insert the spacer 10 into the inter-disc space as shown in FIG. 6, moving the first end 12 of the spacer 10 across the inter-disc space 72 between the medullary portions 66 of the two adjacent vertebrae 60, 62 and across the imaginary front-to-back axial plane 82 to the opposite-from-point-of-entry side until the first nub 18 is located between the apophyseal rings 68 of the adjacent vertebrae 60, 62 as shown in FIG. 7. The insertion tool 80 shown in phantom in FIG. 6 may be threaded into the threaded opening 50 to help insert the spacer 10. A cannula may be used, if desired. The first shoulder 20 has a height that is greater than the inter-disc space between the adjacent apophyseal rings 68, so it will prevent the spacer 10 from being pushed out beyond the apophyseal ring 68 on the opposite-from-point-of-entry side.

Next, if an insertion tool 80 is used, it is released from the spacer 10. Then the second end 14 of the spacer 10 is repositioned by pushing forward on or near the second end 14 to rotate the spacer 10 so it pivots about the first nub 18 until the second nub 34 lies in the inter-disc space between the apophyseal rings 68 on the point-of-entry side, as shown in FIG. 8. The second shoulder 36 has a height that is greater than the inter-disc space between the apophyseal rings 68, so it will prevent the spacer 10 from being pushed out beyond the apophyseal ring 68 on the point-of-entry side of the vertebrae. If the spacer is pushed far enough, the respective shoulders 20, 36 will abut the inner surfaces of the apophyseal rings 68, which will serve as a stop.

Once the spacer 10 is in the desired position, with the nubs 18, 34 located in the inter-disc space between the apophyseal rings 68, release the set screws from the rod to allow the vertebrae 60, 62 to fall back onto the spacer 10, as seen in FIGS. 9 and 10, so the top and bottom surfaces of the nubs 18, 34 contact the apophyseal rings 68 of the upper and lower vertebrae 60, 62, respectively, and the convex central portion 16 of the spacer 10 contacts or at least very closely approaches the medullary portions of the two adjacent vertebrae in order to encourage bone growth and fusion.

Then re-tighten the set screws on the opposite-from-point-of-entry side and install a similar set of pedicle screws, rod, and set screws on the point-of-entry side of the vertebrae 60, 62.

In its final location, the spacer 10 lies with the first and second ends 12, 14 of the spacer 10 lying on opposite sides of the front-to-back axis 82, and with the central portion 16 of the spacer 10 lying in the medullary portion of the inter-disc space 72 between the two adjacent vertebrae, and with the first and second nubs 18, 34 in contact with and supporting the apophyseal rings 68 of the upper and lower vertebrae 60, 62. The central portion 16 of the spacer 10 is generally convex and generally conforms to the shape of the inter-disc space 72 between the medullary portions of the two adjacent vertebrae. Preferably, the central portion 16 also contacts the vertebrae to facilitate bone growth for fusing the bones together.

It may be noted that a similar procedure could be used to insert the spacer 10 from the other side of the spinal column. In that case, the spacer 10 would simply be flipped end-over-end before insertion.

FIG. 16 is a broken away, perspective view of the right rear portion of another embodiment of a spacer, which is identical to the first embodiment, except that this embodiment includes a small opening 51 on the nub 34, adjacent the opening 50 but located along the rear surface ‘of the spacer. This opening 51 may be identical to the opening 50, including internal threads for receiving the end of the insertion tool 80 (See FIG. 6). Alternatively, it may be a simple hemispherical depression (or some other indentation) to provide a purchase for the end of an insertion tool to push against when repositioning the second end of the spacer to the position shown in FIG. 8. It will be obvious to those skilled in the art that modifications may be made to the embodiments described above without departing from the scope of the present invention as claimed. For instance, FIG. 12 depicts an alternate embodiment of a spacer 10′ wherein the top and bottom nub surfaces 42′, 44′ respectively are not parallel to each other as they are in the embodiment 10 shown in FIG. 11. The degree of offset from a truly parallel orientation of the top and bottom nub surfaces may be chosen to approximate the degree of natural curvature of the spine at the point of implantation of the spacer 10′.

What is claimed is:

1. A spacer for spacing apart two adjacent vertebrae, comprising:
an elongated body having top, bottom, front and rear body surfaces and defining openings through said top and bottom body surfaces extending into a hollow interior;
said elongated body extending in a horizontal elongated direction from a first end to a second end and defining a body height between said top and bottom body surfaces, including defining a maximum body height, wherein the distance in the horizontal elongated direction from the first end to the second end is greater than the maximum body height; and
including first and second nubs projecting outwardly in said horizontal elongated direction at said first and second ends, respectively, each of said nubs having top and bottom nub surfaces and defining a nub height between said top and bottom nub surfaces, including defining a maximum nub height that does not exceed 8 mm;
wherein each of said nubs extends in the elongated direction for a distance of between two and six_millimeters and the maximum nub heights of the first and second nubs are substantially equal;
wherein the maximum height of said elongated body is at least four millimeters greater than the maximum nub heights;
wherein at least one of the top and bottom surfaces of said elongated body defines a first shoulder recessed from said first end, with a concave inflection point defining an intersection between said first nub and said first shoulder.

2. A spacer for spacing apart two adjacent vertebrae as recited in claim 1, wherein at least one of the top and bottom surfaces of said elongated body defines a second shoulder recessed from said second end, with a concave inflection point defining an intersection between said second nub and said second shoulder; and wherein said elongated body includes a central portion between said first and second shoulders, and the top and bottom surfaces of said central portion are substantially convex.

3. A spacer for spacing apart two adjacent vertebrae as recited in claim 2, wherein the first and second nub top surfaces are substantially coplanar and the first and second nub bottom surfaces are substantially coplanar.

4. A spacer for spacing apart two adjacent vertebrae as recited in claim 3, wherein each of the first and second nub top surfaces and first and second nub bottom surfaces defines a plane.

5. A spacer for spacing apart two adjacent vertebrae as recited in claim 4, wherein the plane defined by the nub top surfaces is substantially parallel to the plane defined by the nub bottom surfaces.

6. A spacer for spacing apart two adjacent vertebrae as recited in claim 3, and further defining at least one opening through said front body surface to the hollow interior, wherein said rear body surface defines less than half as much open area as said front body surface.

7. A spacer for spacing apart two adjacent vertebrae as recited in claim 3, wherein said rear body surface has less than ten percent open area.

8. A spacer for spacing apart two adjacent vertebrae as recited in claim 2, said elongated body being sized and shaped so as to include means for the first and second nubs to fit between and abut the apophyseal rings of two adjacent vertebrae to maintain the desired spacing between the two adjacent vertebrae, with the central portion of the elongated body fitting between and generally conforming to the shape of the space between the medullary portions of the two adjacent vertebrae.

9. A spacer for spacing apart two adjacent vertebrae as recited in claim 8, and further comprising a threaded opening at the second end of said elongated body and an elongated installation tool having a threaded end that mates with said threaded opening.

10. A spacer for spacing apart two adjacent vertebrae as recited in claim 2, and further comprising a threaded opening at the second end of said elongated body and an elongated installation tool having a threaded end that mates with said threaded opening.

11. A method of spinal fusion for fusing two adjacent, lower and upper vertebrae, each vertebra including an apophyseal ring and a medullary core portion encircled by the apophyseal ring, wherein said two adjacent vertebrae define an inter-disc space between them, and wherein each vertebra defines an imaginary front-to-back axis which bisects the vertebra into first and second substantially mirror-image halves, comprising the steps of:
inserting the first end of an elongated spacer having a first end, a second end, and a central portion lying between said first and second ends into the inter-disc space between the apophyseal rings of the two adjacent vertebrae by entering into the inter-disc space at a point within 45 degrees of the front-to-back axis; then
moving the first end of the spacer across the inter-disc space between the medullary portions of the two adjacent vertebrae and into the inter-disc space between the apophyseal rings of the two adjacent vertebrae until the first end of the spacer contacts the apophyseal ring of at least a first of the two adjacent vertebrae and lies in the inter-disc space between the apophyseal rings of the two adjacent vertebrae; and
positioning the second end of the spacer so that it also contacts the cortical apophyseal ring of the first of the two adjacent vertebrae and lies in the inter-disc space between the apophyseal rings of the two adjacent vertebrae, with the first and second ends of the spacer lying on opposite sides of the front-to-back axis, and with the central portion of the spacer lying in the inter-disc space between the medullary portions of the two adjacent vertebrae.

12. A method of spinal fusion as recited in claim 11, wherein said first end of said spacer defines a first shoulder recessed from said first end and a first nub which projects from said first shoulder, said first nub having a smaller height than the central portion, and wherein the step of moving the first end of the spacer across the inter-disc space between the medullary portions of the two adjacent vertebrae and then into the inter-disc space between the apophyseal rings of the two adjacent vertebrae includes the step of causing said first shoulder on said spacer to abut the interior of the apophyseal ring of said first of the two adjacent vertebrae thereby stopping the progress of the first end of said spacer.

13. A method of spinal fusion as recited in claim 12, wherein the central portion of the spacer is generally convex and generally conforms to the shape of the inter-disc space between the medullary portions of the two adjacent vertebrae.

14. A method of spinal fusion as recited in claim 13, wherein the step of positioning the second end includes pivoting said spacer by pushing on said second end of said spacer.

15. A method of spinal fusion as recited in claim 14, wherein said second end of said spacer defines a second shoulder recessed from said second end, and a second nub which projects from said second shoulder, and further comprising the step of causing said second shoulder to abut the interior of said first of the two adjacent vertebrae thereby stopping the progress of the second end of said spacer.

16. A method of spinal fusion as recited in claim 12, wherein said entry into the inter-disc space is through the superior and inferior facets of the lower and upper adjacent vertebrae, respectively.

17. A method of spinal fusion as recited in claim 16, and further comprising the steps of:
removing a sufficient amount of the facets on the entry side of the two adjacent vertebrae to provide a point of entry;
cutting the ligamentum Flavum at the point of entry;
distracting the two adjacent vertebrae on the opposite-from-entry side to achieve a first distraction;
securing the two adjacent vertebrae to maintain said first distraction; then
distracting the two adjacent vertebrae on the entry side, then releasing the two adjacent vertebrae, and then further distracting the two adjacent vertebrae on the entry side to a second distraction; and then, inserting said spacer.
